SEQUENCE LISTING (1 sequence)

SEQ ID NO: 1 - protein, 5 aa, Artificial Sequence
  Peptidyl ghrelin analog
  MOD_RES (1)...(1): Xaa = isonipecotic acid (Inp)
  MOD_RES (2)...(2): Xaa = D-3-benzothienylAla (D-Bal)
  MOD_RES (3)...(3): Xaa = D-Trp
  MOD_RES (5)...(5): Xaa = Apc
  AMIDATION (5)...(5): NH2
XXXFX

(19) United States
(12) Patent Application Publication
(10) Pub. No.: US 2020/0172572 A1
(43) Pub. Date: Jun. 4, 2020

(54) PROCESS FOR THE LIQUID PHASE SYNTHESIS OF H-INP-(D)BAL-(D)TRP-PHE-APC-NH2, AND PHARMACEUTICALLY ACCEPTABLE SALTS THEREOF

(71) Applicant: ALLERGAN GI CORP., Madison, NJ (US)

(72) Inventors: Karel Decroos, Sint-Amandsberg (BE); Olivier Titeux, Limelette (BE)

(21) Appl. No.: 16/788,934

(22) Filed: Feb. 12, 2020

Related U.S. Application Data
(63) Continuation of application No. 15/123,050, filed on Sep. 1, 2016.
(60) Provisional application No. 61/947,748, filed on Mar. 4, 2014.

(51) Int. Cl. C07K 5/107; C07K 14/60; C07K 5/065; C07K 7/06; C07K 5/097; C07K 5/078

(52) U.S. Cl. CPC C07K 5/1016; C07K 14/60; C07K 5/06139; C07K 7/06; C07K 5/0821; C07K 5/06078

(57) ABSTRACT

The present invention provides a process for the liquid phase synthesis of the Ghrelin analog H-Inp-(D)Bal-(D)Trp-Phe-Apc-NH₂ (SEQ ID NO: 1, Formula (I)), pharmaceutically acceptable salts thereof.

### RELATED APPLICATION

This application is a Continuation of U.S. application Ser. No. 15/123,050, filed Sep. 1, 2016, which is a U.S. National Phase Application under 35 U.S.C. § 371 of International Application No. PCT/US2015/018589, filed Mar. 4, 2015, which claims the benefit of and priority to U.S. Provisional Application No. 61/947,748, filed Mar. 4, 2014, the contents of which are incorporated herein by reference in its entirety.

### BACKGROUND OF THE INVENTION

Ghrelin is a 28 amino acid peptide hormone produced by the gut that plays a central role in feeding regulation, nutrient absorption, GI motility and energy homeostasis. The secretion of ghrelin increases under conditions of negative energy balance—during starvation, cachexia, and anorexia nervosa—while its expression decreases under conditions of positive energy balance—during feeding, hyperglycemia, and obesity. It is the endogenous ligand for the growth hormone secretagogue receptor (GHSR) and the GHSR-1a which asserts at least some part of its function through activation of the GHSR-1a including stimulation of growth hormone secretion under selected physiological conditions.

Ghrelin analogs have a variety of different therapeutic uses (see, e.g., U.S. Pat. Nos. 7,456,253 and 7,932,231, the entire contents of which are incorporated herein by reference.

A particularly therapeutically promising Ghrelin analog is H-Inp-D-Bal-D-Trp-Phe-Apc-NH₂ (Formula (I), SEQ ID NO: 1). To date, this analog has been prepared only by solid phase synthesis. There is a need for liquid phase synthesis approaches that provide acceptable scale up manufacturing of the Ghrelin analog is H-Inp-D-Bal-D-Trp-Phe-Apc-NH₂ (SEQ ID NO: 1), and pharmaceutically acceptable salts thereof. For example, liquid phase procedures providing a desirable yield, high purity (e.g., stereochemical purity), cost efficiency or a combination thereof are needed.

### SUMMARY OF THE INVENTION

The present invention provides novel processes for the synthesis of the Ghrelin analog H-Inp-(D)Bal-(D)Trp-Phe-Apc-NH₂ (SEQ ID NO: 1), and pharmaceutically acceptable salts thereof, which can be advantageously used to scale up the synthesis of the Ghrelin analog H-Inp-D-Bal-D-Trp-Phe-Apc-NH₂ (SEQ ID NO: 1).

In one embodiment, the present invention is a process for the synthesis of a peptide of Formula (I)

H-Inp-(D)Bal-(D)Trp-Phe-Apc-NH₂, (I)

or pharmaceutically acceptable salt thereof. The process comprises at least one step of coupling any two amino acids of the peptide of Formula (I) in a liquid phase.

In another embodiment, the present invention is a peptide fragment of structural formula (II)

Boc-Inp-(D)Bal-(D)Trp-Phe-Apc(Boc)-NH₂, (II)

or a salt thereof.

In another embodiment, the present invention is a peptide fragment of structural formula (III)

Boc-Inp-DBal-DTrp-OH, (III)

or a salt thereof.

In another embodiment, the present invention is a peptide fragment of structural formula (IV)

H-Phe-Apc(Boc)-NH₂, (IV)

or a salt thereof.

In another embodiment, the present invention is a peptide fragment of structural formula (V)

H-DBal-DTrp-OH, (V)

or a salt thereof.

In another embodiment, the present invention is a peptide fragment of structural formula (VI)

Z-Phe-Apc(Boc)-NH₂, (VI)

or a salt thereof.

The methods of liquid phase peptide synthesis disclosed herein possess a number of advantages. For example, the liquid phase synthetic method disclosed herein provides for a convergent rather than a stepwise synthetic scheme, thereby improving total yield. Furthermore, employing silylating agents advantageously allows for the use of aprotic organic solvents, thus avoiding the disadvantages of aqueous solvents such as formation of deletion impurities. Employing the silylated intermediates further permits the use of backbone-unprotected amino acid residues as intermediates, thus reducing the number of synthetic steps and improving yield. A further advantage of the disclosed method is found in performing the amidation of the N-terminal amino acid residue (Apc) at a dipeptide stage rather than as at single amino acid residue stage. Such amidation results in reduction of ammonia contamination and, subsequently, avoiding premature peptide chain termination due to aminolysis of the activated carbocylic group by the dissolved ammonia.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a block-diagram illustrating a sequence of steps employed by an example embodiment of a method disclosed herein.

FIG. 2 is a block-diagram illustrating a sequence of steps employed by an example embodiment of a method disclosed herein.

FIG. 3 is a block-diagram illustrating a sequence of steps employed by an example embodiment of a method disclosed herein.

FIG. 4 is a block-diagram illustrating a sequence of steps employed by an example embodiment of a method disclosed herein.

FIG. 5 is a block-diagram illustrating a sequence of steps employed by an example embodiment of a method disclosed herein.

FIG. 6 is a block-diagram illustrating a sequence of steps employed by an example embodiment of a method disclosed herein.

FIG. 7 is a block-diagram illustrating a sequence of steps employed by an example embodiment of a method disclosed herein.

FIG. 8 is a block-diagram illustrating a sequence of steps employed by an example embodiment of a method disclosed herein.

FIG. 9 is a block-diagram illustrating a sequence of steps employed by an example embodiment of a method disclosed herein.

FIG. 10 is a block-diagram illustrating a sequence of steps employed by an example embodiment of a method disclosed herein.

FIG. 11 is a block-diagram illustrating a sequence of steps employed by an example embodiment of a method disclosed herein.

FIG. 12 is a block-diagram illustrating a sequence of steps employed by an example embodiment of a method disclosed herein.

FIG. 13 is a block-diagram illustrating a sequence of steps employed by an example embodiment of a method disclosed herein.

FIG. 14 is a block-diagram illustrating a sequence of steps employed by an example embodiment of a method disclosed herein.

FIG. 15 is a block-diagram illustrating a sequence of steps employed by an example embodiment of a method disclosed herein.

FIG. 16 is an illustration of a synthetic scheme employed by an example embodiment of a method disclosed herein to produce an intermediate useful for practicing the present invention.

FIG. 17 is an illustration of a synthetic scheme employed by an example embodiment of a method disclosed herein to produce an intermediate useful for practicing the present invention.

FIG. 18 is an illustration of a synthetic scheme employed by an example embodiment of a method disclosed herein to produce an intermediate useful for practicing the present invention.

FIG. 19 is an illustration of a synthetic scheme employed by an example embodiment of a method disclosed herein to produce an intermediate useful for practicing the present invention.

FIG. 20 is an illustration of a synthetic scheme employed by an example embodiment of a method disclosed herein to produce an intermediate useful for practicing the present invention.

FIG. 21 is an illustration of a synthetic scheme employed by an example embodiment of a method disclosed herein to produce an intermediate useful for practicing the present invention.

FIG. 22 is an illustration of a synthetic scheme employed by an example embodiment of a method disclosed herein to produce an intermediate useful for practicing the present invention.

FIG. 23 is an illustration of a synthetic scheme employed by an example embodiment of a method disclosed herein to produce an intermediate useful for practicing the present invention.

FIG. 24 is an illustration of a synthetic scheme employed by an example embodiment of a method disclosed herein to produce the compound of Formula (I).

### DETAILED DESCRIPTION OF THE INVENTION

The nomenclature used to define the peptides is that typically used in the art wherein the amino group at the N-terminus appears to the left and the carboxyl group at the C-terminus appears to the right.

As used herein, the term “amino acid” includes both a naturally occurring amino acid and a non-natural amino acid. The term “amino acid,” unless otherwise indicated, includes both isolated amino acid molecules (i.e. molecules that include both, an amino-attached hydrogen and a carbonyl carbon-attached hydroxyl) and residues of amino acids (i.e. molecules in which either one or both an amino-attached hydrogen or a carbonyl carbon-attached hydroxyl are removed). The amino group can be alpha-amino group, beta-amino group, etc. For example, the term “amino acid alanine” can refer either to an isolated alanine H-Ala-OH or to any one of the alanine residues H-Ala-, -Ala-OH, or -Ala-. Unless otherwise indicated, all amino acids found in the compounds described herein can be either in D or L configuration. The term “amino acid” includes salts thereof, including pharmaceutically acceptable salts. Any amino acid can be protected or unprotected. Protecting groups can be attached to an amino group (for example alpha-amino group), the backbone carboxyl group, or any functionality of the side chain. As an example, phenylalanine protected by a benzyloxycarbonyl group (Z) on the alpha-amino group would be represented as Z-Phe-OH.

As used herein, the term “peptide fragment” refers to two or more amino acids covalently linked by at least one amide bond (i.e. a bond between an amino group of one amino acid and a carboxyl group of another amino acid selected from the amino acids of the peptide fragment). The terms “polypeptide” and “peptide fragments” are used interchangeably. The term “peptide fragment” includes salts thereof, including pharmaceutically acceptable salts.

As used herein, the term “coupling” refers to a step of reacting two chemical moieties to form a covalent bond. When referring to coupling of amino acids, the term “coupling” means a step of reacting two amino acids, thereby forming a covalent amide bond between an amino group of one amino acid residue and a carboxyl group (e.g., the backbone carboxyl group) of another amino acid.

As used herein, the term “carboxyl activating group” means a group that modifies a carboxyl group of an amino acid or a carboxyl terminus of a peptide fragment to be susceptible to aminolysis. Commonly, a carboxyl activating group is an electron withdrawing moiety that substitutes the hydroxyl moiety of a carboxyl group. Such electron withdrawing moiety enhances polarization and thereby the electrophilicity at the carbonyl carbon. As used herein, the term “activated carboxyl group” refers to a carboxyl group in which the hydroxyl group has been replaced by a carboxyl activating group.

As used herein, the term “nucleophilic additive” means a chemical compound or unit that is used in an organic synthesis in order to control its stereochemical outcome.

As used herein, the term “silylated amino acid” refers to an amino acid that has been modified by a silyl-containing moiety at least one modifiable position. Examples of modifiable positions include —NH and —OH functional groups. Such modification is the result of reacting an amino acid with a silylating agent, as described below. In an example embodiment, a silylated amino acid is persilylated, i.e. modified by a silyl-contaning moiety at all modifiable positions.

To facilitate the large scale synthesis of the Ghrelin analog H-Inp-(D)Bal-(D)Trp-Phe-Apc-NH₂ (SEQ ID NO: 1), novel processes for its synthesis are provided herein. Generally, the entire process is carried out in solution phase, that is, without solid phase reactions such as the coupling of an amino acid with a resin bound amino acid.

There is a growing body of evidence to support a separate Ghrelin pathway that has some overlap with GHSR-1a and which increases weight and GI motility, without the release of GH The most compelling evidence is derived from ghrelin peptide analogs that are complete antagonists of GH SR-h, and do not stimulate GH release but affect GI motility and increase body weight.

Pharmacology studies with the Ghrelin analog H-Inp-(D)Bal-(D)Trp-Phe-Apc-NH₂ (SEQ ID NO: 1), a small peptide ghrelin agonist, and human clinical trials conducted with full-length human ghrelin in cancer, cardiac and COPD cachexias demonstrate increases in appetite, weight and cardiac output without apparent toxicity. Given the potent pro-kinetic effects of ghrelin, GI motility disorders are also targeted clinical applications for a ghrelin agonist, particularly post-operative ileus, opioid-induced constipation, gastroparesis, irritable bowel syndrome and chronic constipation. Ghrelin and the Ghrelin analog H-Inp-(D)Bal-(D)Trp-Phe-Apc-NH₂ (SEQ ID NO: 1) also possesses anti-inflammatory properties, suppressing a range of inflammatory cytokines, so that GI Inflammatory conditions such as Inflammatory Bowel Disease are additional potential clinical targets.

A description of example embodiments of the invention follows.

A first embodiment of the present invention is a process for the synthesis of H-Inp-(D)Bal-(D)Trp-Phe-Apc-NH₂, or pharmaceutically acceptable salt thereof, comprising coupling amino acids in liquid phase.

A second embodiment of the present invention is a process for the synthesis of H-Inp-(D)Bal-(D)Trp-Phe-Apc-NH₂, or pharmaceutically acceptable salt thereof, comprising preparing a silylated amino acid by silylating an unprotected or protected amino acid or unprotected or protected peptide fragment by reaction with a silylating agent in a polar aprotic organic solvent.

A protected amino acid is an amino acid in which one or more functional groups are protected with a protecting group. A protected peptide fragment is a dipeptide, tripeptide, or tetrapeptide, in which one or more functional groups of the amino acid of the peptide fragment are protected with a protecting group. Preferably, the protected amino acid and/or protected peptide fragment of the present invention have a protected amino group. The term “amino protecting group” refers to protecting groups which can be used to replace an acidic proton of an amino group in order to reduce its nucleophilicity.

Examples of amino protecting groups (e.g. X¹, X², X³, X⁴, etc.) include but are not limited to substituted or unsubstituted groups of acyl type, such as the formyl, acrylyl (Acr), benzoyl (Bz), acetyl (Ac), trifluoroacetyl, substituted or unsubstituted groups of aralkyloxycarbonyl type, such as the benzyloxycarbonyl (Z), p-chlorobenzyloxycarbonyl, p-bromobenzyloxycarbonyl, p-nitrobenzyloxycarbonyl, p-methoxybenzyloxycarbonyl, benzhydryloxycarbonyl, 2(p-biphenylyl)isopropyloxycarbonyl, dimethoxyphenyl)isopropyloxycarbonyl, p-phenylazobenzyloxycarbonyl, triphenylphosphonoethyloxycarbonyl or 9-fluorenylmethyloxycarbonyl group (Fmoc), substituted or unsubstituted groups of alkyloxycarbonyl type, such as the tert-butyloxycarbonyl (BOC), tert-amyloxycarbonyl, diisopropylmethyloxycarbonyl, isopropyloxycarbonyl, ethyloxycarbonyl, allyloxycarbonyl, 2 methylsulphonylethyloxycarbonyl or 2,2,2-trichloroethyloxycarbonyl group, groups of cycloalkyloxycarbonyl type, such as the cyclopentyloxycarbonyl, cyclohexyloxycarbonyl, adamantyloxycarbonyl or isobornyloxycarbonyl group, and groups containing a hetero atom, such as the benzenesulphonyl, p-toluenesulphonyl, mesitylenesulphonyl, methoxytrimethylphenylsulphonyl, 2-nitrobenzenesulfonyl, 2-nitrobenzenesulfenyl, 4-nitrobenzenesulfonyl or 4-nitrobenzenesulfenyl group. Among these groups X, those comprising a carbonyl, a sulfenyl or a sulphonyl group are preferred. An amino protecting groups X¹, X², X³, X⁴, etc. is preferably selected from allyloxycarbonyl groups, tert-butyloxycarbonyl (BOC), benzyloxycarbonyl (Z), 9 fluorenylmethyloxycarbonyl (Fmoc), 4-nitrobenzenesulfonyl (Nosyl), 2-nitrobenzenesulfenyl (Nps) and substituted derivatives.

Preferred amino protecting groups X¹, X², X³, X⁴, etc. for the process of the present invention are tert-butyloxycarbonyl (Boc), a 9-fluorenylmethyloxycarbonyl (Fmoc), and a benzyloxy-carbonyl (Z). Even more preferred amino protecting groups for the process of the present invention are tert-butyloxycarbonyl (Boc) and a benzyloxy-carbonyl (Z).

Amino protecting groups X¹, X², X³, X⁴, etc. can be introduced by various methods as known in the art. For example, by reaction with suitable acid halides or acid anhydrides. On the other hand, amino protecting groups X¹, X², X³, X⁴, etc. can be removed (i.e., the step of deprotecting), for example, by acidolysis, hydrogenolysis (e.g., in the presence of hydrogen (e.g. bubbled through the liquid reaction medium) and catalyst such as palladium catalyst), treatment with dilute ammonium hydoxide, treatment with hydrazine, treatment with sodium and treatment with sodium amide.

In a preferred embodiment, the process according to any one of the embodiments described herein, is carried out without protecting the carboxyl groups of the amino acids. Each amino acid coupling step of the synthesis comprises coupling of an amino acid having a protected amino group and optionally an activated carboxyl group with an amino acid having an unprotected amino group and an unprotected carboxyl group.

Preferably, silylating an unprotected or protected amino acid or unprotected or protected peptide fragment includes the silylating of an unprotected amino group of the unprotected or protected amino acid or unprotected or protected peptide fragment.

The silylated fragment prepared in a process of the present invention (e.g., the process of the second embodiment) can be isolated and purified if desired; however, it is preferred to use the silylated fragment in situ.

Typical silylating agents include N,O-bis(trimethylsilyl)acetamide, N,O-bis(trimethylsilyl)trifluoroacetamide, hexamethyldisilazane, N-methyl-N-trimethylsilylacetamide, N,-methyl-N-trimethylsilyltrifluoroacetamide, N-(trimethylsilyl)acetamide, N-(trimethylsilyl)diethylamine, N-(trimethylsilyl)dimethylamine, 1-(trimethylsilyl)imidazole, 3-(trimethylsilyl)-2-oxazolidone, and (trimethylsilyl)-N-dimethyl-acetamide. The preferred silylating agent is (trimethylsilyl)-N-dimethyl-acetamide.

The silylating reactions of the present invention are generally carried out at a temperature from 0° C. to 100° C., and preferably from 25° C. to 50° C.

Generally 0.5 to 5, preferably 0.7 to 3, more preferably 1 to 2.5, and even more preferably about 2 or 1.8 to 2.2 equivalent of silylating agent are used relative to the molar amount of amino groups to be silylated.

Generally the silylation of the present invention is carried out in the presence of a polar aprotic organic solvent. More typically the solvent is an aprotic organic solvent having a static relative permittivity of between 5 and 10. Preferably, the solvent is ethyl acetate.

In a third embodiment of the present invention, the process of any one of the described embodiments comprises reacting a silylated fragment (e.g., the silylated fragment of the second embodiment) with (1) a protected and activated amino acid or (2) a protected and activated peptide fragment, having an amino protecting group and an activated carboxyl group.

Generally the reaction of the silylated fragment (e.g., the silylated fragment of the second or third embodiment) with (1) a protected and activated amino acid or (2) a protected and activated peptide fragment, having an amino protecting group and an activated carboxyl group, is carried out in the presence of a polar aprotic organic solvent. More typically the solvent is an aprotic organic solvent having a static relative permittivity of between 5 and 10. Preferably, the solvent is ethyl acetate.

Typically, the reaction solution used for silylating, and/or used in the subsequent amino acid or peptide coupling reaction of the silylated fragment, contains from 10% wt to 90% wt or polar aprotic solvent relative to the total weight of the solution.

Generally, the reaction of a silylated fragment of the present invention with (1) a protected and activated amino acid or (2) a protected and activated peptide fragment, the amino acid or petide fragment having an amino protecting group and an activated carboxyl group, is carried out at a temperature from −50° C. to 50° C.

Suitable carboxyl group activating agents (also referred to herein as “activators”) include, but are not limited to, N-hydroxysuccinimide (HOSu), N-hydroxyphthalimide, pentafluorophenol (PfpOH), and di-(p-chlorotetrafluorophenyl)carbonate. As known in the are these activators form active esters. Preferably, the activator is N-hydroxysuccinimide (HOSu).

In a preferred embodiment of the present invention, the process for the synthesis of the Ghrelin analog makes use of X¹-(D)Bal-OSu, X⁴-Inp-OSu, and X³-Phe-OSu, wherein each X¹, X³, and X⁴, independently, is an amino protecting group.

In a fourth embodiment of the present invention, the process of any one of the embodiments described herein, further includes silylating the amino acid H-(D)Trp-OH to form a silylated residue of the amino acid H-(D)Trp-OH, and reacting the silylated residue of the amino acid H-(D)Trp-OH with an amino acid X¹-(D)Bal-Y¹, wherein X¹ is an amino protecting group, and Y¹ is an activated carboxyl group. In a particular embodiment, X¹ is Boc and Y¹ is -OSu. In a more particular embodiment, X¹ is Boc and Y¹ is -OSu, and the silylating and coupling reactions are carried out each in ethyl acetate.

In a fifth embodiment of the present invention, the process of any one of the embodiments described herein, further includes silylating an amino acid H-Apc(X²)—OH to form a silylated residue of the amino acid H-Apc(X²)—OH and reacting the silylated residue of the amino acid H-Apc(X²)—OH with an amino acid X³-Phe-Y², wherein X² is an amino protecting group, and Y² is an activated carboxyl group. X³ is as defined above. In a particular embodiment, H-Apc(X²)—OH is H-Apc(Boc)-OH and X³-Phe-Y² is Z-Phe-OSu. In a more particular embodiment, H-Apc(X¹)—OH is H-Apc(Boc)-OH and X²-Phe-Y³ is Z-Phe-OSu, and the silylating and coupling reactions are each carried out in ethyl acetate.

A sixth embodiment of the present invention is a process of any one of the embodiments described herein, wherein the fragment X³-Phe-Apc(X²)—NH₂ is prepared by coupling a silylated residue of an amino acid H-Apc(X²)—OH and an amino acid X³-Phe-Y² in an organic solvent, followed by carboxyl group amidation. In a particular embodiment, the amino acid H-Apc(X²)—OH is silylated in ethyl acetate by reacting it with (trimethylsilyl)-N-dimethyl-acetamide. In a more particular embodiment, a suspension of H-Apc(X²)—OH, ethyl acetate and (trimethylsilyl)-N-dimethyl-acetamide is formed, the suspension heated (to 35° C. to 50° C.; preferably, about 45° C.), and after substantial completion of silylation, X³-Phe-Y² is added. Preferably, X³-Phe-Y-2 is Z-Phe-OSu and H-Apc(X²)—OH is H-Apc(Boc)-OH. Also preferably, the carboxyl group amidation is achieved in the presence of ammonia and DCC. Furthermore preferably, X³-Phe-Y² is Z-Phe-OSu and H-Apc(X²)—OH is H-Apc(Boc)-OH, and the carboxyl group amidation is achieved in the presence of ammonia and DCC.

A seventh embodiment of the present invention is a process of any one of the embodiments described herein, wherein the peptide fragment X⁴-Inp-(D)Bal-(D)Trp-OH is prepared from the peptide fragment H-(D)Bal-(D)Trp-OH and X⁴-Inp-Y³ in the presence of a base, wherein Y³ is an activated carboxyl group. In a particular embodiment, the base is diisopropylethylamine, X⁴ is Boc, and Y³ is -OSu. In a further particular embodiment, HCl.H-(D)Bal-(D)Trp-OH is solubilised at 10° C. to 70° C. (preferably, about 40° C.) in an organic solvent (e.g., DMA) in the presence of a base to form a solution, the solution is subsequently cooled (e.g. to 0° C.), and Boc-Inp-OSu is added to the solution at 10° C. to 30° C.

An eighth embodiment of the present invention is a process of any one of the embodiments described herein, further comprising preparing X⁴-Inp-(D)Bal-(D)Trp-Phe-Apc(X²)—NH2 from X⁴-Inp-(D)Bal-(D)Trp-OH and H-Phe-Apc(X²)—NH2 in the presence of a nucleophilic additive and a coupling reagent. In a particular embodiment the nucleophilic additive is HOPO. In another particular embodiment the nucleophilic additive is HOPO and the coupling reagent is EDC. In yet another particular embodiment, H-Phe-Apc(X²)—NH2, X⁴-Inp-(D)Bal-(D)Trp-OH, and the nucleophilic additive are solubilized in an organic solvent, and subsequently, the coupling reagent is added. In yet another particular embodiment, H-Phe-Apc(X²)—NH2, X⁴-Inp-(D)Bal-(D)Trp-OH, and HOPO are solubilized in an organic solvent at 10° C. to 30° C. (preferably, about 25° C.) to form a solution, the solution is cooled (e.g., to 2° C. to 10° C.), and subsequently, the EDC is added. Preferably, H-Phe-Apc(X²)—NH2 is H-Phe-Apc(Boc)-NH2, and X⁴-Inp-(D)Bal-(D)Trp-OH is Boc-Inp-(D)Bal-(D)Trp-OH. Further preferably, the organic solvent is dimethylacetamide. In another particular embodiment, the process further comprises synthesizing Boc-Inp-(D)Bal-(D)Trp-Phe-Apc(Boc)-NH2 by reacting Boc-Inp-(D)Bal-(D)Trp-OH and H-Phe-Apc(Boc)-NH2 in an organic solvent and in the presence of 2-hydroxypyridine-N-oxide and 1-(3-dimethylaminopropyl)-3-ethylcarbodiimide.

A ninth embodiment of the present invention is a process of any one of the embodiments described herein, further comprising deprotecting Z-Phe-Apc(Boc)-NH2 by hydrogenolysis to form H-Phe-Apc(Boc)-NH2. In a particular embodiment, Z-Phe-Apc(Boc)-NH2 is solubilised in an organic solvent (e.g. methanol), and deprotecting includes adding catalyst (e.g., palladium catalyst) to the organic solvent and flowing or generating hydrogen in the organic solvent. Preferably, the organic solvent is methanol.

A ninth embodiment of the present invention is a process of any one of the embodiments described herein, further comprising deprotecting Boc-Inp-(D)Bal-(D)Trp-Phe-Apc(Boc)-NH2 by acidolysis to obtain H-Inp-(D)Bal-(D)Trp-Phe-Apc-NH2.2HCl. In a particular embodiment, the acidolysis is carried out in the presence of 4-methylthiophenyl and HCl in isopropanol.

A tenth embodiment of the present invention is a process for the liquid phase synthesis of the Ghrelin analog H-Inp-(D)Bal-(D)Trp-Phe-Apc-NH₂, or pharmaceutically acceptable salt thereof, comprising (a) synthesizing fragment H-(D)Bal-(D)Trp-OH from silylated H-(D)Trp-OH and X¹-(D)Bal-Y¹ in an organic solvent, (b) synthesizing fragment X³-Phe-Apc(X²)—NH2 from silylated H-Apc(X²)—OH and X³-Phe-Y⁴ in an organic solvent, (c) synthesizing fragment X⁴-Inp-(D)Bal-(D)Trp-OH from H-(D)Bal-(D)Trp-OH and X⁴-Inp-Y³ in an organic solvent and in the presence of a base, and (d) synthesizing X-Inp-(D)Bal-(D)Trp-Phe-Apc(X²)—NH2 from X⁴-Inp-(D)Bal-(D)Trp-OH and H-Phe-Apc(X²)—NH2 in the presence of a nucleophilic additive and a coupling reagent. In particular embodiments, one or more of the steps (a), (b), (c) and (d) of the tenth embodiment can be performed, independently, as described above for the first to ninth embodiment, including as described in the respective particular and preferred embodiments. In further particular embodiments, one or more of the steps (a), (b), (c) and (d) can be performed as described in the respective exemplification below. Preferably, each coupling reagent, independently, is a carbodiimide reagent.

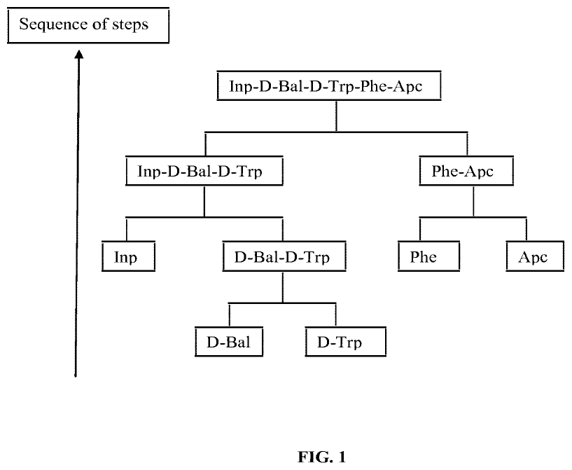

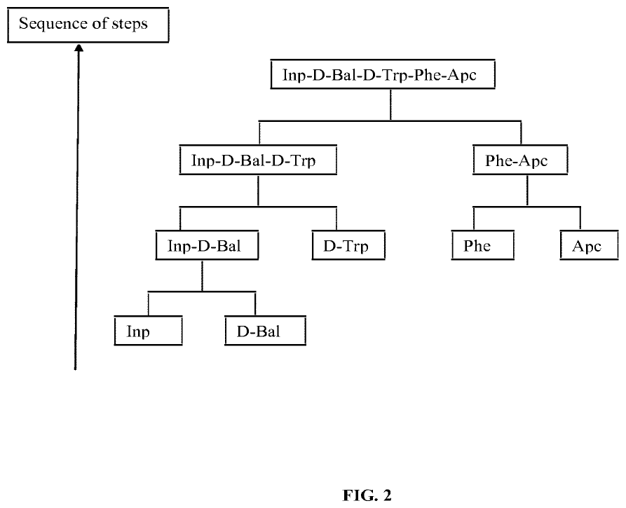

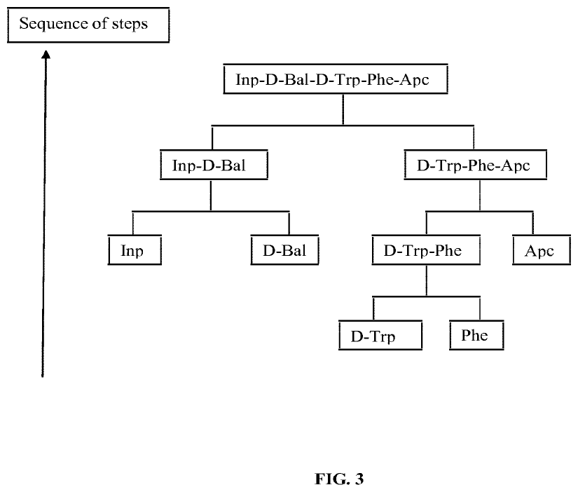

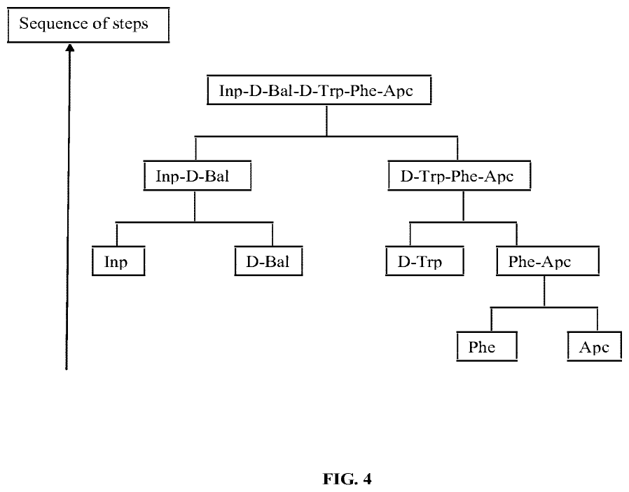

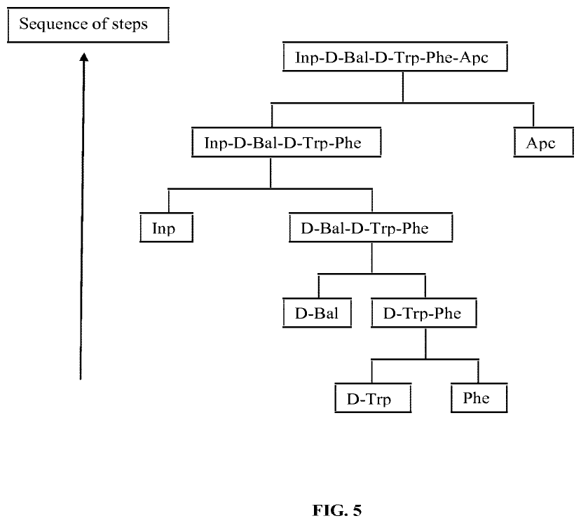

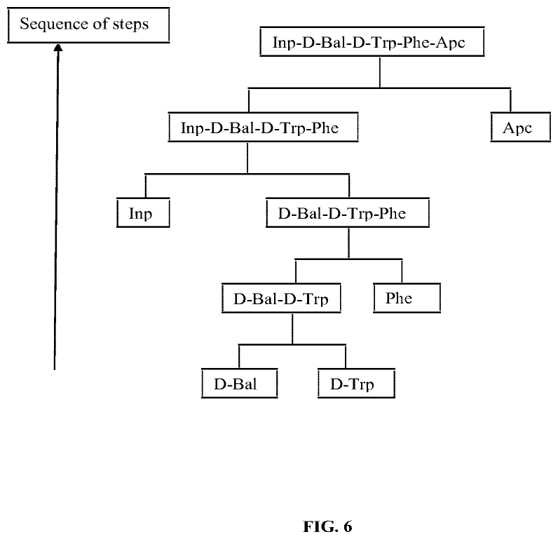

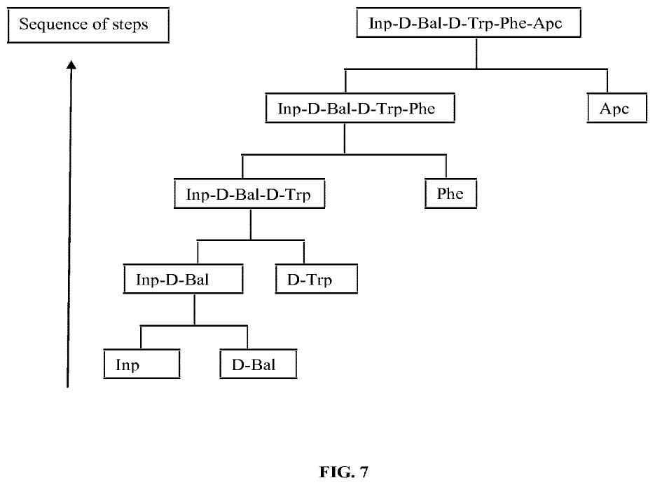

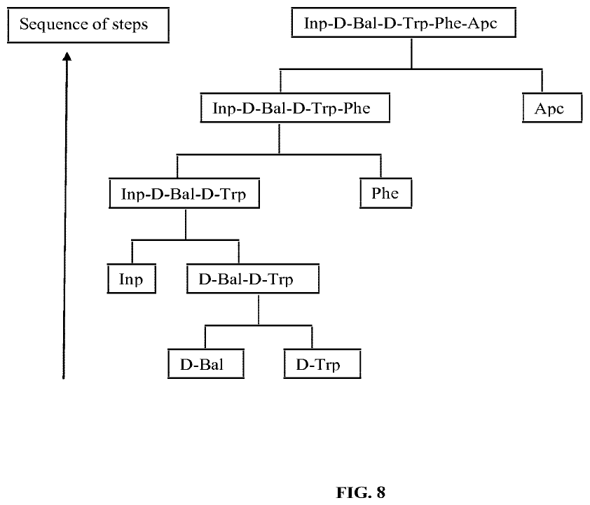

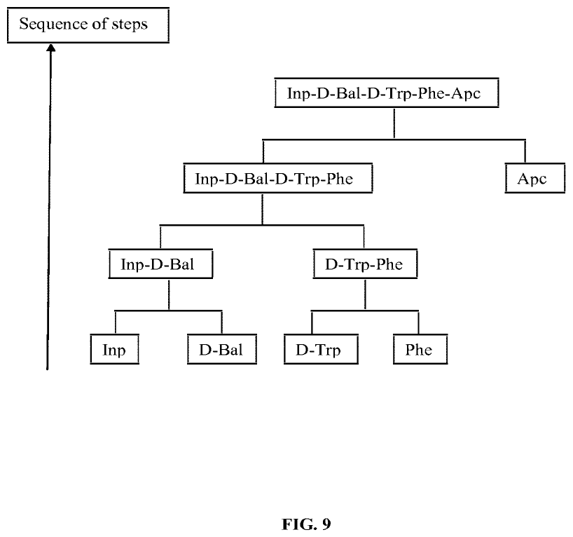

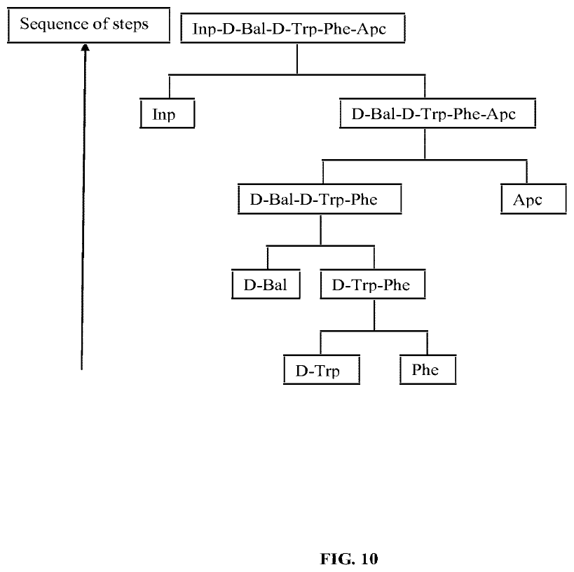

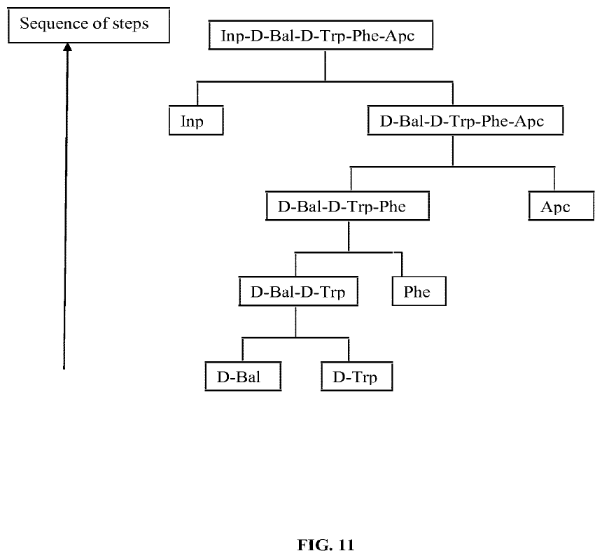

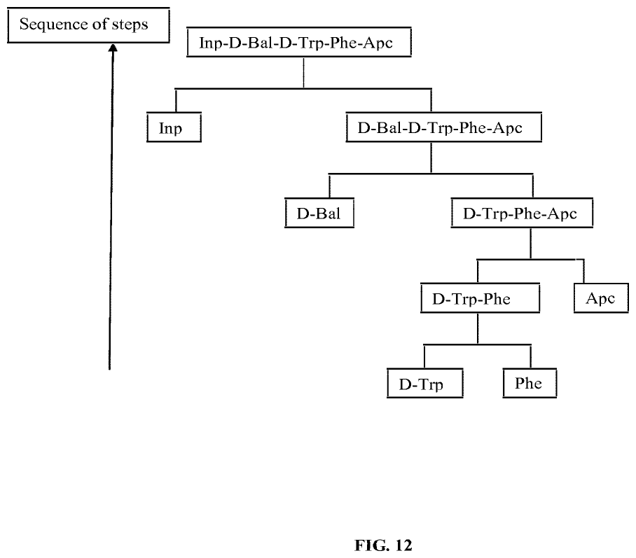

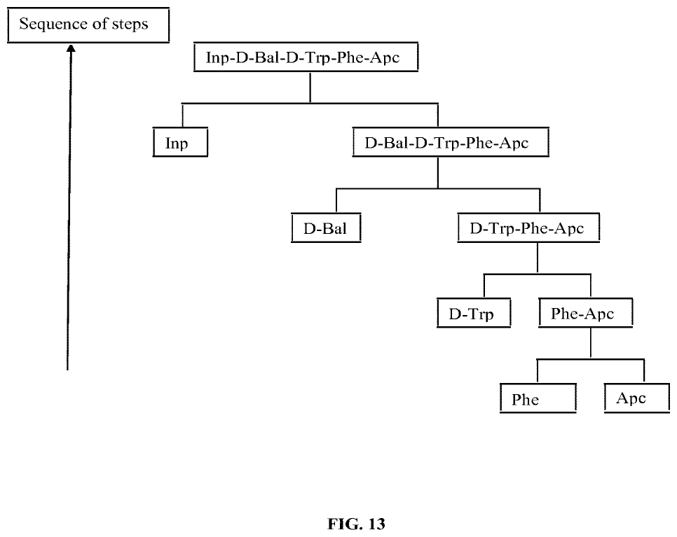

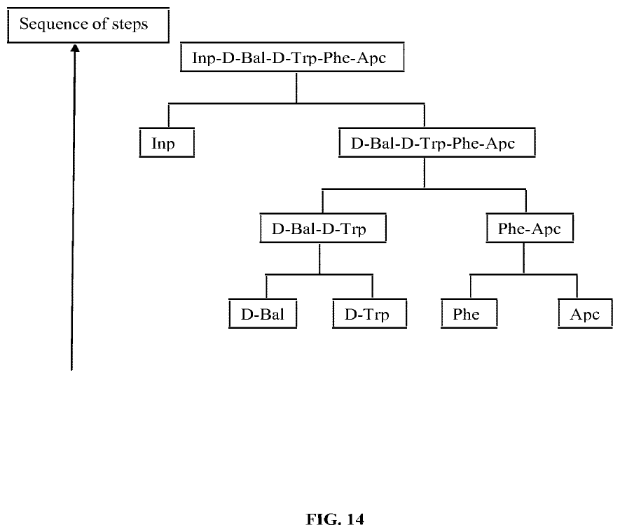

Further embodiments for the liquid phase synthesis of the Ghrelin analog H-Inp-(D)Bal-(D)Trp-Phe-Apc-NH₂ (SEQ ID NO: 1), or pharmaceutically acceptable salt (e.g., acetate salt of H-Inp-(D)Bal-(D)Trp-Phe-Apc-NH₂) are schematically diagrammed in FIGS. 1 to 14, including linear synthesis as shown in FIGS. 7 and 12, and convergent synthesis in FIGS. 1-6, 8-11, 13 and 14. Convergent syntheses are preferred, and particularly, the synthesis as schematically shown in FIG. 1 is preferred. The amino groups of the amino acids and peptide fragments shown in FIGS. 1 to 14 can be protected as described herein, preferably, with amino protecting groups Boc and Z, carboxyl groups can be activated as described herein (e.g., with HOSu), and these amino acids and peptide fragments can be coupled in the sequence as shown in each of FIGS. 1-14 with the coupling reagents and with the coupling reactions as described herein.

The Ghrelin analog H-Inp-(D)Bal-(D)Trp-Phe-Apc-NH₂ (SEQ ID NO: 1), or pharmaceutically acceptable salt (e.g., hydrochloride sale of H-Inp-(D)Bal-(D)Trp-Phe-Apc-NH₂) can be further purified and lyophilized to obtain a lyophilized Ghrelin analog. Accordingly, a further embodiment of the present invention is process for preparing a lyophilized Ghrelin analog, the process comprising preparing a crude product comprising H-Inp-(D)Bal-(D)Trp-Phe-Apc-NH₂, or a pharmaceutically acceptable salt thereof, according to any one of the embodiments described herein, and further comprising purifying the crude product by high-performance liquid chromatography to obtain a purified product, and lyophilizing the purified product to obtain the lyophilized Ghrelin analog. In a particular embodiment, the process comprises eluting the crude product on a column (preferably, of C18-grafted silica) with an acetonitrile/ammonium acetate buffer gradient to obtain an eluate, fractionating the eluate, pooling fractions of desired purity (e.g., >95%) to obtain a pooled fraction, diluting the pooled fraction with water to obtain a diluted pooled fraction, eluting the diluted pooled fraction with acetonitrile-rich gradient to obtain a second eluate, fractionating the second eluate, pooling second fractions of desired purity to obtain a pooled high purity fraction, evaporating acetonitrile under vacuum from the pooled high purity fraction to obtain an aqueous solution, and freeze-drying the aqueous solution to obtain the lyophilized Ghrelin analog.

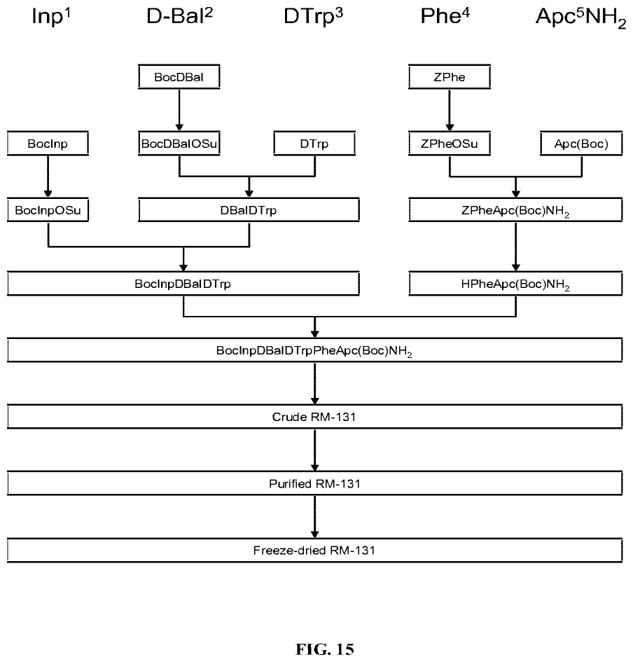

FIG. 15 is a schematic diagram for the preparation of a lyophilized Ghrelin analog of the sequence H-Inp-(D)Bal-(D)Trp-Phe-Apc-NH₂ (SEQ ID NO: 1), including synthetic sequences for the synthesis of the Ghrelin analog H-Inp-(D)Bal-(D)Trp-Phe-Apc-NH₂ (SEQ ID NO: 1).

Coupling reagents of the present invention are typically carbodiimide reagents. Examples of carbodiimide reagents include, but are not limited to, N,N′-dicyclohexylcarbodiimide (DCC), 1-(3-dimethylaminopropyl)-3-ethylcarbodiimide (EDC), N-cyclohexyl-N′-isopropylcarbodiimide (CIC), N,N′-diisopropylcarbodiimide (DIC), N-tert-butyl-N′-methylcarbodiimide (BMC), N-tert-butyl-N′-ethylcarbodiimide (BEC), bis[[4-(2,2-dimethyl-1,3-dioxolyl)]-methyl]carbodiimide (BDDC), and N,N-dicyclopentylcarbodiimide. DCC is a preferred coupling reagent.

Nucleophilic additives of the present invention typically are selected from the group consisting of 2-hydroxypyridine-N-oxide (HOPO), 1-hydroxy-7-azabenzotriazole (HOAt), 1-hydroxy-benzotriazole (HOBt), 3,4-dihydro-3-hydryoxy-4-oxo-1,2,3-benzotriazine (HODhbt), and ethyl-1-hydroxy-1 H-1,2,3-triazole-4-carboxylate (HOCt).

Typically, the silylating agent of the present invention is selected from the group consisting of N,O-Bis(trimethylsiliyl)acetamide, N,O-Bis(trimethylsilyl)trifluoroacetamide, Hexamethyldisilazane, N-Methyl-N-trimethylsilylacetamide, N-Methyl-N-trimethylsilyltrifluoroacetamide, Trimethylchlorosilane+base, N-(Trimethylsilyl)acetamide, Trimethylsilyl cyanide, N-(Trimethylsilyl)dietylamine, N-(Trimethylsilyldimethylamine, 1-(Trimethylsilyl)imidazole, and 3-Trimethyldilyl-2-oxazolidinone. In an example embodiment, the silylating agent is (trimethylsilyl)-N-dimethyl-acetamide.

The processes described herein, typically, can further include reaction quenching steps (e.g., by the addition of 3-(dimethylamino)propylamine), washing steps (e.g., with organic solvent (e.g., acetonitrile, diisopropylether, isopropanol, or cyclohexane), with a solution of KHSO₄ (e.g., 4 (w/v) % solution of KHSO₄), with a solution of NaCl (e.g., 2 (w/v) % solution of NaCl), with demineralised water, with a solution of NaHCO₃ (e.g., 4 (w/v) % solution of NaHCO₃)), concentrating steps (e.g., concentrating under vacuum, crystallizing, filtering, precipitating), and drying steps (e.g., drying under vacuum or azeotropic distillation).

The nomenclature used to define the peptides is that typically used in the art wherein the amino group at the N-terminus appears to the left and the carboxyl group at the C-terminus appears to the right.

As used herein, the term “amino acid” includes both a naturally occurring amino acid and a non-natural amino acid.

Certain amino acids present in compounds of the invention can be and are represented herein as follows:

Ape denotes the following structure corresponding to 4-aminopiperidine-4-carboxylic acid:

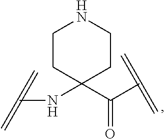

Bal denotes the following structural formula corresponding to 3-benzothienylalanine:

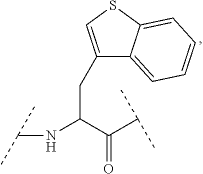

Inp denotes the following structural formula corresponding to isonipecotic acid:

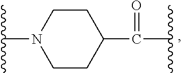 [SMILES: *C(=O)C1CCN(*)CC1]

Phe denotes the following structural formula corresponding to phenylalanine:

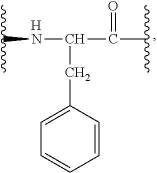

and

Trp denotes the following structural formula corresponding to tryptophan:

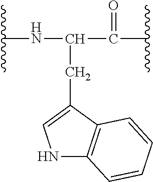 [SMILES: *NC(Cc1c[nH]c2ccccc12)C(*)=O]

Certain other abbreviations used herein are defined as follows:

BDDC is bis[[4-(2,2-dimethyl-1,3-dioxolyl)]-methyl]carbodiimide,

BEC is N-tert-butyl-N′-ethylcarbodiimide,

BMC is N-tert-butyl-N′-methylcarbodiimide,

Boc is tert-butyloxycarbonyl,

CIC is N-cyclohexyl-N′-isopropylcarbodiimide;

DMA is dimethylamine,

DCC is N,N′-dicyclohexylcarbodiimide

DCU is N,N′-dicyclohexylurea

DIC is N,N′-diisopropylcarbodiimide,

DIEA or DIPEA is diisopropylethylamine,

EDC is 1-(3-dimethylaminopropyl)-3-ethylcarbodiimide,

Fmoc is fluorenylmethyloxycarbonyl,

HOAt is 1-hydroxy-7-azabenzotriazole,

HOBt is 1-hydroxy-benzotriazole,

HOCt is ethyl-1-hydroxy- 1H-1,2,3-triazole-4-carboxylate,

HODhbt is 3,4-dihydro-3-hydryoxy-4-oxo-1,2,3-benzotriazine,

HOPO is 2-hydroxypyridine-N-oxide,

HOSu or SucOH is N-hydroxysuccinimide,

PfpOH is pentafluorophenol, and

Z is benzyloxycarbonyl.

With the exception of the N-terminal amino acid, all abbreviations of amino acids (for example, Phe) in this disclosure stand for the structure of —NH—C(R)(R′)—CO—, wherein R and R′ each is, independently, hydrogen or the side chain of an amino acid (e.g., R=benzyl and R′=H for Phe), or R and R′ may be joined to form a ring system as is the case for Ape and Inp. Accordingly, 4-aminopiperidine-4-carboxylic acid is H-Apc-OH, 3-benzothienylalanine is H-Bal-OH, isonipecotic acid is H-Inp-OH, phenylalanine is H-Phe-OH, and tryptophan is H-Trp-OH. The designation “OH” for these amino acids, or for peptides (e.g., Boc-Inp-(D)Bal-(D)Trp-OH) indicates that the C-terminus is the free acid. The designation “NH₂” in, for example, for intermediate, protected dipeptide Z-Phe-Apc(Boc)-NH₂ or for peptide H-Inp-(D)Bal-(D)Trp-Phe-Apc-NH₂ indicates that the C-terminus of the protected peptide fragment is amidated. Further, certain R and R′, separately, or in combination as a ring structure, can include functional groups that require protection during the liquid phase synthesis, for example, the R and R′ group of Apc, can be protected with a further group, for example, a Boc group: Apc(Boc). Yet further, the N-terminus of the amino acids can be protected with an amine protecting group X such as Boc leading to the following denotation: X-Inp-OH, X-Bal-OH, etc (e.g, Boc-Inp-OH, Boc-Bal-OH, etc.). The carboxyl group of the amino acids can be activated, for example, with an activator Y such as N-hydroxysuccinimide (HOSu) leading to the following denotation H-Inp-Y (e.g., H-Inp-OSu).

Where the amino acid has isomeric forms, it is the L form of the amino acid that is represented unless otherwise explicitly indicated as D form, for example, (D)Bal or D-Bal.

The ghrelin analog H-Inp-DBal-D-Trp-Phe-Apc-NH₂ (SEQ ID NO: 1) can be prepared as acidic or basic salts. Pharmaceutically acceptable salts (in the form of water- or oil-soluble or dispersible products) include conventional non-toxic salts or the quaternary ammonium salts that are formed, e.g., from inorganic or organic acids or bases. Examples of such salts include acid addition salts such as acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate; and base salts such as ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine and lysine. Preferably, the ghrelin analog H-Inp-DBal-DTrp-Phe-Apc-NH₂ (SEQ ID NO: 1) is prepared as an acetate salt.

### EXEMPLIFICATION

### Synthesis of H-Inp-(D)Bal-(D)Trp-Phe-Apc-NH₂ According to the Scheme Shown in FIG. 15

The below described syntheses make use of (1) protected amino acids as starting material, specifically, Boc-Inp-OH, Boc-(D)Bal-OH, Z-Phe-OH, and H-Apc(Boc)-OH, and (2) the unprotected amino acid H-(D)Trp-OH. These amino acids are commercially available or can be synthesized with methods known in the art.

### 1. Synthesis of Boc-Inp-OSu

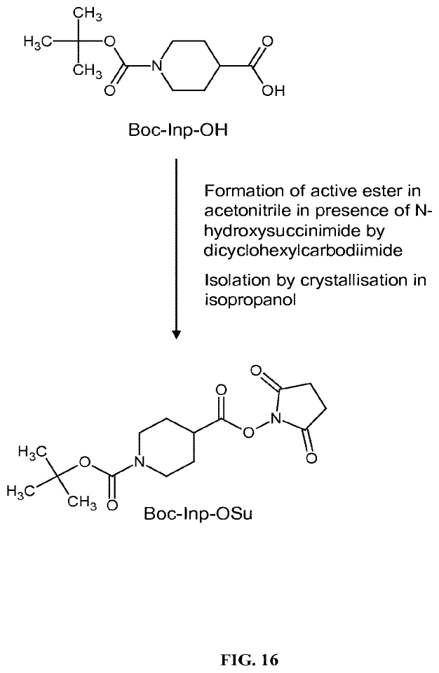

Boc-Inp-OSu was synthesized according to the synthetic scheme shown in FIG. 16.

Specifically, Boc-Inp-OH (1.15 g, 5 mmol) and N-hydroxysuccinimide (SucOH) (0.69 g, 6 mmol) were solubilised in 12.3 mL acetonitrile at room temperature. Once the solids had dissolved, the solution was cooled to 0° C. and DCC (1.08 g 5.25 mmol) dissolved in 1.4 mL acetonitrile was added dropwise. The temperature was controlled at 0° C. for one hour and than gradually increased to room temperature over 4 hours. After overnight reaction, DCC (0.10 g, 0.5 mmol) dissolved in 0.15 ml acetonitrile was added in two portions. Once the reaction was complete the formed DCC was removed by filtration and washed with twice 3.8 ml acetonitrile. The mother liquors were combined and concentrated under vacuum to a solution with a volume of 5 ml. Subsequently, this concentrated solution was added to 10.4 ml isopropanol which provoked precipitation of Boc-Inp-OSu. The suspension was concentrated under vacuum to 8 mL and then diluted with 12.5 ml isopropanol. The solid was filtered-off, washed twice with 3.8 ml isopropanol and dried under vacuum at 45° C. to give 1.51 g of white powder (90% yield).

### 2. Synthesis of Boc-(D)Bal-OSu

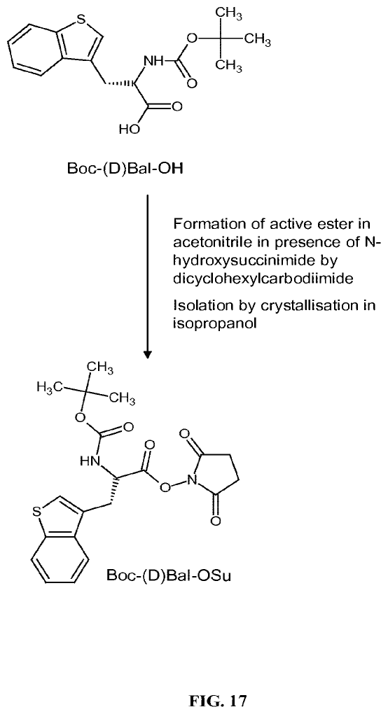

Boc-DBal-OH was synthesized according to the synthetic scheme shown in FIG. 17.

Specifically, Boc-(D)Bal-OH (1.61 g, 5 mmol) and N-hydroxysuccinimide (SucOH) (0.69 g, 6 mmol) were solubilised in 17.6 mL acetonitrile at room temperature. Once the solids dissolved, the solution was cooled to 0° C. and DCC (1.03 g 5 mmol) dissolved in 1.3 mL acetonitrile was added dropwise. The temperature was controlled at 0° C. for one hour and was than gradually increased to room temperature over 4 hours. After overnight reaction, DCC (0.10 g, 0.5 mmol) dissolved in 0.15 ml acetonitrile was added in two portions. Once the reaction was complete the formed DCC was removed by filtration and washed twice with 12 ml acetonitrile. The mother liquors were combined and concentrated under vacuum to a solution with a volume of 13 ml. Subsequently, this concentrated solution was added to 27 mL isopropanol. During further concentration under vacuum Boc-(D)Bal-OSu crystallised. Acetonitrile was further stripped with 43 mL of additional isopropanol. The final volume of the suspension was 53 mL. The solid was filtered-off, washed twice with 9 ml isopropanol followed by 9 ml diisopropylether and dried under vacuum at 45° C. to give 1.83 g of white powder (85% yield).

### 3. Synthesis of H-(D)Bal-(D)Trp-OH

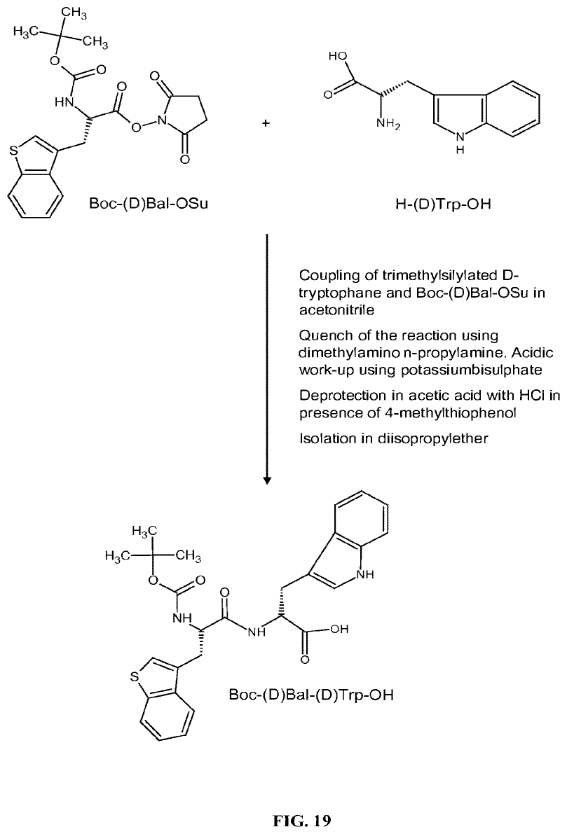

H-(D)Bal-(D)Trp-OH was synthesized according to the synthetic scheme shown in FIG. 19.

Specifically, H-(D)Trp-OH (0.91 g, 4.34 mmol) was added to (trimethylsilyl)-N-dimethyl-acetamide (1.27 g, 8.67 mmol) and 4.1 ml ethyl acetate. The reaction medium was heated at 45° C. until a solution was obtained (in approximately 2 h). The solution was cooled to 0° C. and added to a cold solution of Boc-(D)Bal-OSu (1.83 g 4.25 mmol) in 17.6 mL ethyl acetate. 15 min after the addition, the reaction medium was brought to room temperature. Once the desired conversion rate was obtained (approximately 5 h) the reaction was quenched with 3-(dimethylamino)propylamine (0.11 g 1.06 mmol), followed by two washings with 14.5 ml of a solution of 4 (w/v) % KHSO₄ and one washing with 17 ml of a solution of 2 (w/v) % NaCl and one final washing with 14 mL demineralised water. The resulting organic phase was concentrated under vacuum, 13.4 ml glacial acetic acid was added and the solution was further concentrated to a final volume of 9.7 mL. 4-Methylthiophenol (1.82 g 12.75 mmol) and 4N HCl in dioxane (2.23 g 8.5 mmol) were added. After 2 h the reaction was terminated and the reaction medium was precipitated in 106 mL diisopropylether. The solid was filtered-off and washed twice with 20 ml diisopropylether. After overnight drying under vacuum at 45° C. 1.98 of HCl H-(D)Bal-(D)Trp-OH was obtained (90% yield).

### 4. Synthesis of Boc-Inp-(D)Bal-(D)Trp-OH

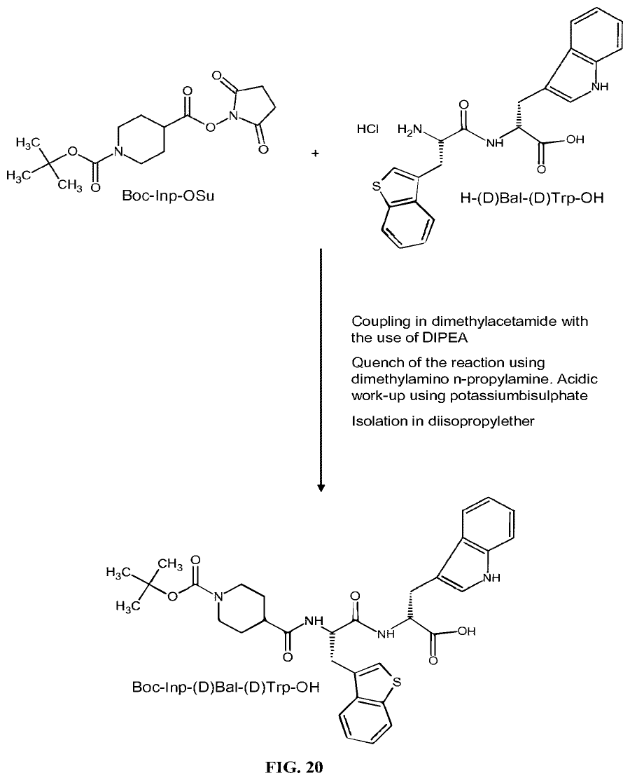

Boc-Inp-(D)Bal-(D)Trp-OH was synthesized according to the synthetic scheme shown in FIG. 20.

Specifically, HCl H-(D)Bal-(D)Trp-OH (1.74 g 3.83 mmol) was solubilised at 40° C. in 13.8 mL DMA in the presence of DIPEA (1.03 g 7.86 mmol). Once a solution had been obtained, the mixture was cooled to 0° C. and Boc-Inp-OSu (1.31 g 4.02 mmol) was added as a solid to this solution at room temperature. One hour after the addition the reaction medium was brought to room temperature. After overnight reaction the conversion was complete and the reaction was quenched by the addition of 3-(dimethylamino)propylamine (0.08 g 0.8 mmol). Subsequently the mixture was diluted with 56 mL ethyl acetate and washed three times with 28 mL of a 4 (w/v) % solution of KHSO₄, followed by one washing with 25 mL demineralised water. The resulting organic phase was concentrated under vacuum and dried by azeotropic distillation. In total 68 mL additional ethyl acetate were added. The solution was concentrated to a final volume of 14 ml and precipitated in 128 mL diisopropylether. The solid was filtered-off, washed twice with 24 mL diisopropylether and dried under vacuum at 45° C. to yield 1.6 g of solid (81% yield).

### 5. Synthesis of Z-Phe-OSu

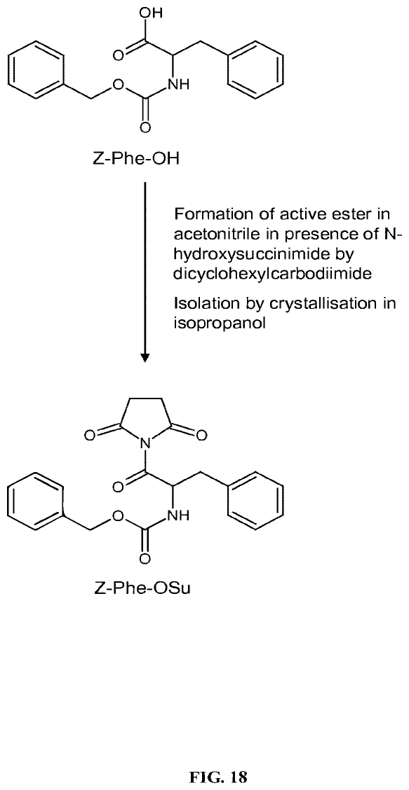

Z-Phe-OSu was synthesized according torn the synthetic scheme shown in FIG. 18.

Specifically, Z-Phe-OH (1.53 g, 5 mmol) and N-hydroxysuccinimide (SucOH) (0.69 g, 6 mmol) were solubilised in 16.3 mL acetonitrile at room temperature. Once the solids had dissolved, the solution was cooled to 0° C. and DCC (1.08 g 5.25 mmol) dissolved in 1.3 mL acetonitrile was added dropwise. The temperature was controlled at 0° C. for one hour and was than gradually increased to room temperature over 4 hours. After overnight reaction, DCC (0.10 g, 0.5 mmol) dissolved in 0.15 ml acetonitrile was added in two portions. Once the reaction was complete the formed DCC was removed by filtration and washed twice with 4 ml acetonitrile. The mother liquors were combined and concentrated under vacuum to a solution with a volume of 6 ml. Subsequently, this concentrated solution was added to 12 mL isopropanol. During further concentration under vacuum Z-Phe-OSu crystallised. Acetonitrile was further stripped with 14.5 mL of additional isopropanol. The final volume of the suspension was 24 mL. The solid was filtered-off, washed twice with 4 ml isopropanol and dried under vacuum at 45° C. to give 1.7 g of white powder (87% yield).

### 6. Synthesis of Z-Phe-Apc(Boc)-NH2

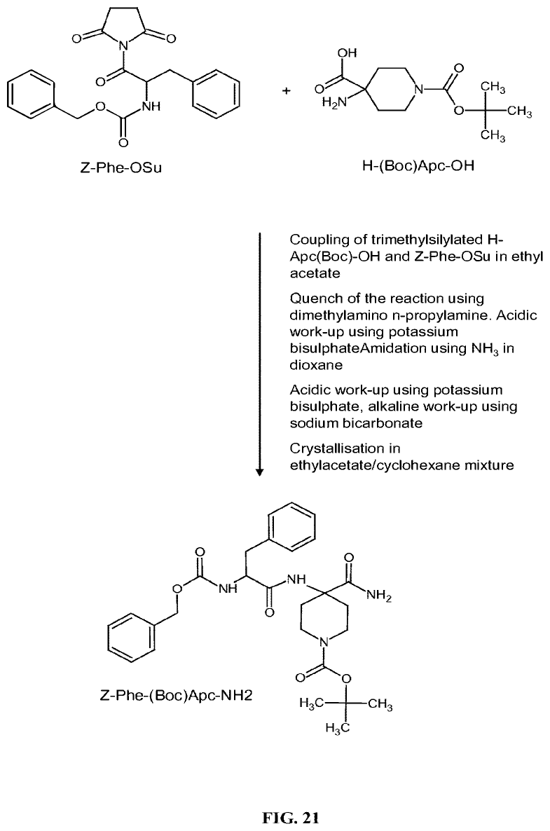

Z-Phe-Apc(Boc)-NH2 was synthesized according to the synthetic scheme shown in FIG. 21.

Specifically, H-Apc(Boc)-OH (1.06 g 4.2 mmol) was added to 8.8 mL ethyl acetate with (trimethylsilyl)-N-dimethyl-acetamide (1.23 g 8.4 mmol). The suspension was heated to 45° C. Once a solution was obtained, a solution of Z-Phe-OSu (1.7 g 4.28 mmol) in 16.1 mL ethyl acetate was added. The temperature was kept at 45° C. and after overnight reaction the reaction was quenched with of 3-(dimethylamino)propylamine (0.11 g 1.07 mmol). Subsequently the mixture was washed twice with 11 mL of a 4 (w/v) % solution of KHSO₄, then with 11 ml of a 2 (w/v) % NaCl and finally with 11 mL demineralised water. The washed organic phase was dried by azeotropic distillation with the addition of 28 ml ethylacetate. The solution was concentrated to a final volume of 28.2 mL and cooled to 0° C. DCC (0.78 g 4.63 mmol) previously solubilised in 1 ml ethyl acetate was added followed by the dropwise addition of 9.261 ml of a solution of 0.5M ammonia (4.63 mmol) in dioxane. Once the addition finished, the mixture was brought to room temperature and after one hour the conversion was complete. The reaction was quenched by the addition of 0.83 mL water and heated for 30 min at 35° C. The DCU was removed by filtration and the resulting solution was washed twice with 33 mL of a 4 (w/v) % solution of KHSO₄, 33 ml of a 4 (w/v) % solution of NaHCO₃ and finally with 33 ml demineralised water. The washed organic phase was dried by azeotropic distillation. Therefore, 29 ml of ethyl acetate was added. The final volume was 7.8 mL. To this solution was added 7.7 ml hot cyclohexane. Z-Phe-Apc(Boc)-NH₂ crystallised overnight at 5° C. After filtration of the crystals, washing twice with 15 mL cyclohexane, the solid was dried under vacuum at 45° C. 1.98 g of white crystals were obtained (yield 87%).

### 7. Synthesis of H-Phe-Apc(Boc)-NH2

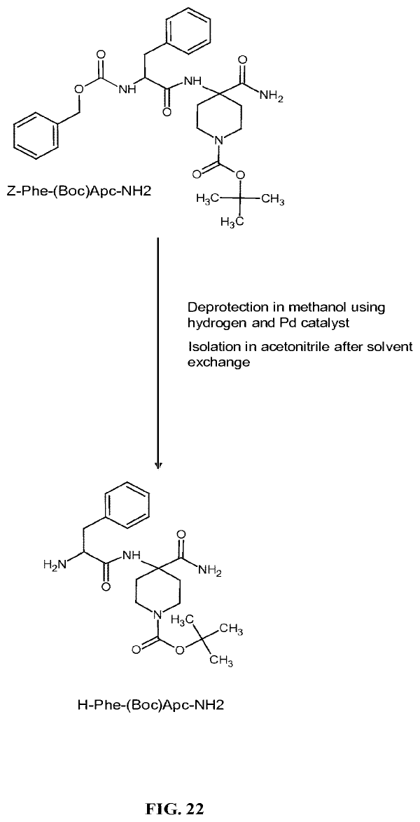

H-Phe-Apc(Boc)-NH2 was synthesized according to the synthetic scheme shown in FIG. 22.

Specifically, Z-Phe-Apc(Boc)-NH₂ (1.97 g 3.65 mmol) was solubilised in 6.15 ml methanol. After addition of 0.194 g of palladium catalyst supported on charchoal (0.18 mmol) the reaction was inerted by N₂ bubbling during 30 min and subsequently hydrogen was bubbled through the solution at 35° C. After 2 h the reaction was complete and the catalyst was filtered-off. The resulting solution was concentrated under vacuum and 9 ml acetonitrile was added. The solution was further concentrated and H-Phe-Apc(Boc)-NH₂ crystallised. When a volume of 4.1 ml was reached the suspension was filtered and the solid was whashed twice with 10 ml diisopropylether. The solid was dried under vacuum at 45° C. and 1.3 g of solid was obtained (yield 87%).

### 8. Synthesis of Boc-Inp-(D)Bal-(D)Trp-Phe-Apc(Boc)-NH2

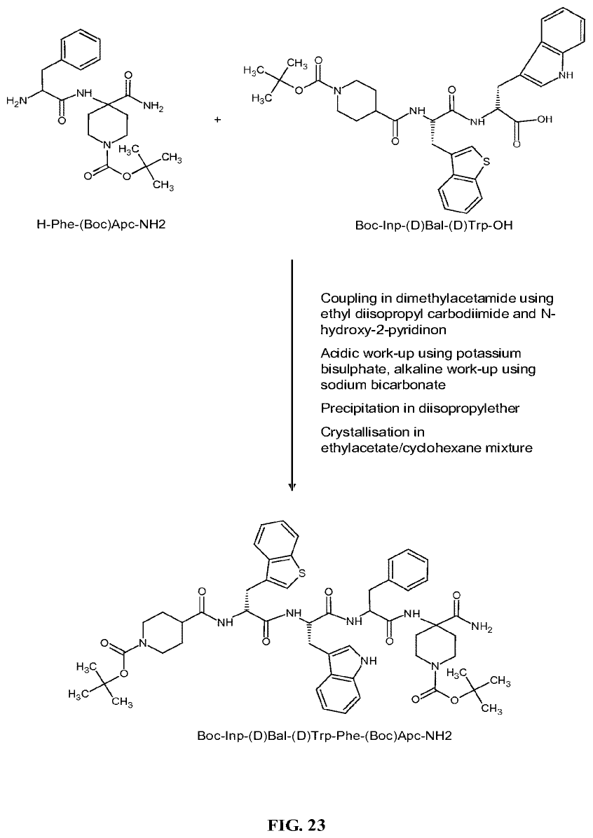

Boc-Inp-(D)Bal-(D)Trp-Phe-Apc(Boc)-NH2 was synthesized according to the synthetic scheme shown in FIG. 23.

Specifically, H-Phe-Apc(Boc)-NH₂ (0.95 g 2.35 mmol), Boc-Inp-(D)Bal-(D)Trp-OH (1.6 g 2.47 mmol) and 2-hydroxypyridine-N-oxide (0.32 g 2.84 mmol) were solubilised in 11.3 mL dimethylacetamide at room temperature. Once a solution was obtained, the mixture was cooled to 5° C. and ethyl-N″-dimethylpropylamine carbodiimide (0.55 g 2.84 mmol) was added. After lh the temperature was set at 10° C., and after 5 h the mixture was heated to room temperature. After overnight reaction a satisfying conversion was obtained and the mixture was diluted with 40 ml ethyl acetate. The resulting solution was washed with 19 mL of a 4 (w/v) % solution of KHSO₄, three times 14 ml of a 4 (w/v) % solution of NaHCO₃ and finally with 15 ml demineralised water. The washed organic phase was dried by azeotropic distillation. Therefore, 29 ml of ethyl acetate was added. The final volume was 16.3 mL. To this solution was added 19 ml hot cyclohexane. Boc-Inp-(D)Bal-(D)Trp-Phe-Apc(Boc)-NH₂ crystallised overnight at 5° C. After filtration of the crystals, washing twice with 15 mL cyclohexane, the solid was dried under vacuum at 45° C. 2 g of white crystals were obtained (yield 86%).

9. Synthesis of H-Inp-(D)Bal-(D)Trp-Phe-Apc-NH2 (crude)

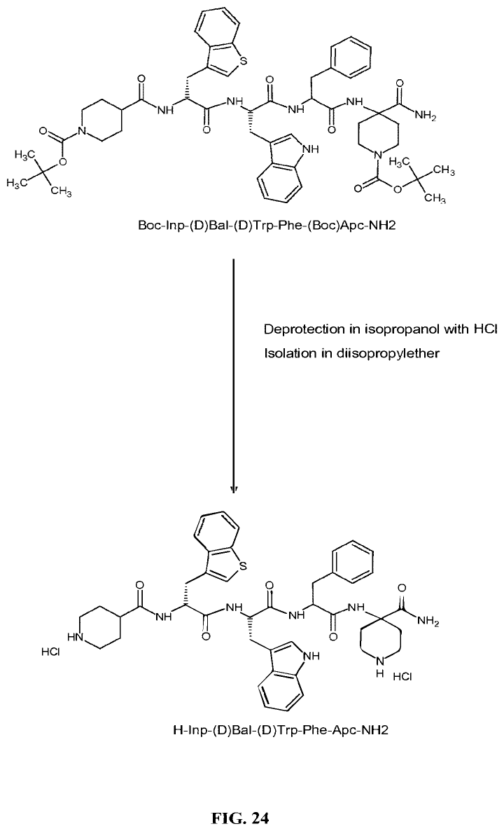

H-Inp-(D)Bal-(D)Trp-Phe-Apc-NH2 was synthesized according to the synthetic scheme shown in FIG. 24.

Specifically, Boc-Inp-(D)Bal-(D)Trp-Phe-Apc(Boc)-NH₂ (2 g 2.02 mmol) and 4-methylthiopenol were solubilised in 9 ml isopropanol. HCl 5N in isopropanol (3.3 ml 20.2 mmol) were added and the mixture was heated at 40° C. After overnight reaction the reaction was complete and a suspension was formed. The suspension was diluted with 83 ml diisopropylether and filtered-off. The solid was washed three times with 10 ml diisopropylether. After drying under vacuum at 45° C., 1.7 g of solid was obtained (70% yield).

10/11. Purification/lyophilisation of the crude H-Inp-(D)Bal-(D)Trp-Phe-Apc-NH2

Crude product was eluted on a column of C18-grafted silica with an acetontrile/ammonium acetate buffer gradient. The eluate was fractionated and fractions with a purity of greater than 95% were pooled. The fractions were diluted with water, charged again on the column and eluted with an acetonitrile-rich gradient. The acetonitrile was evaporated under vacuum and the resulting aqueous solution was freeze-dried to yield the final product, which is the acetate salt of the title polypeptide.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A process for the synthesis of a peptide of Formula (I)
H-Inp-(D)Bal-(D)Trp-Phe-Apc-NH₂, (I)
or pharmaceutically acceptable salt thereof, comprising at least one step of coupling any two amino acids of the peptide of Formula (I) in a liquid phase.

2. The process of claim 1, further comprising a step of reacting a silylating agent with a first amino acid selected form the amino acids of the peptide of Formula (I) in a polar aprotic organic solvent, thereby producing a first silylated amino acid.

3. The process of claim 2, further comprising at least one step of coupling the first silylated amino acid with a second amino acid selected from the amino acids of the peptide of Formula (I).

4. The process of any one of claims 1 to 3, comprising the step of reacting a silylating agent with the amino acid H-(D)Trp-OH in a polar aprotic organic solvent, thereby forming a silylated amino acid residue of the amino acid H-(D)Trp-OH or a salt thereof.

5. The process of claim 4, further comprising the step of reacting the silylated amino acid residue of the amino acid H-(D)Trp-OH with an amino acid of the following formula
X¹-(D)Bal-Y¹,
thereby producing a peptide fragment of the following formula
X¹-(D)Bal-(D)Trp-OH,
or a salt thereof,
wherein X¹ is an amino protecting group, and Y¹ is a carboxyl activating group.

6. The process of any one of claims 1 to 3, comprising the step of reacting a silylating agent with the amino acid H-Apc(X²)—OH in a polar aprotic organic solvent, thereby forming a silylated amino acid residue of the amino acid H-Apc(X²)—OH or a salt thereof, wherein X² is an amino protecting group.

7. The process of claim 6, further comprising the step of reacting the silylated amino acid residue of the amino acid H-Apc(X²)—OH with an amino acid X³-Phe-Y², thereby forming a peptide fragment of the following formula
X³-Phe-Apc(X²)—OH
or a salt thereof,
wherein Y² is a carboxyl activating group, and X³ is an amino protecting group.

8. The process of claim 7, further including the step of reacting the peptide fragment of the following formula
X³-Phe-Apc(X²)—OH,
with an amidating agent, thereby producing a peptide fragment of the following formula
X³-Phe-Apc(X²)—NH₂ or a salt thereof.

9. The process of claim 8, wherein the amidating agent is ammonia.

10. The process of claim 9, further including the step of deprotecting the peptide fragment of the following formula
X³-Phe-Apc(X²)—NH₂,
thereby producing a peptide fragment of the following formula
H-Phe-Apc(X²)—NH₂
or a salt thereof.

11. The process of claim 10, further including the step of deprotecting the peptide fragment of the following structural formula
X¹-(D)Bal-(D)Trp-OH,
thereby producing a peptide fragment of the following formula
H-(D)Bal-(D)Trp-OH
or a salt thereof.

12. The process of claim 11, further comprising the step of reacting an amino acid X⁴-Inp-Y³ with the peptide fragment of the following formula
H-(D)Bal-(D)Trp-OH,
in a liquid solvent, thereby producing a peptide fragment of the following formula
X⁴-Inp-(D)Bal-(D)Trp-OH
or a salt thereof,
wherein X⁴ is an amino protecting group, and Y³ is a carboxyl activating group.

13. The process of claim 12, wherein the liquid solvent is an organic solvent.

14. The process of claim 12, further comprising the step of reacting the peptide fragment of the following formula
X⁴-Inp-(D)Bal-(D)Trp-OH,
with the peptide fragment of the following formula
H-Phe-Apc(X²)—NH₂,
in the presence of a nucleophilic additive,
thereby producing a peptide fragment of the following formula
X⁴-Inp-(D)Bal-(D)Trp-Phe-Apc(X²)—NH₂
or a salt thereof,
wherein X² is an amino protecting group.

15. The process of claim 14, further including the step of deprotecting the peptide fragment of the following formula
X⁴-Inp-(D)Bal-(D)Trp-Phe-Apc(X²)—NH₂,
thereby producing the peptide fragment of Formula (I)
H-Inp-(D)Bal-(D)Trp-Phe-Apc-NH₂ (I)
or a salt thereof.

16. The process of any one of claims 1 to 3, comprising the steps of:
reacting a first silylating agent with the amino acid H-(D)Trp-OH in a first liquid solvent, thereby forming a silylated amino acid residue of the amino acid H-(D)Trp-OH or a salt thereof;
reacting the silylated amino acid residue of the amino acid H-(D)Trp-OH with an amino acid X¹-(D)Bal-Y¹ in a second liquid solvent, thereby producing a peptide fragment of the following formula
X¹-(D)Bal-(D)Trp-OH
or a salt thereof,
wherein X¹ is an amino protecting group, and Y¹ is a carboxyl activating group;
reacting a second silylating agent with the amino acid H-Apc(X²)—OH in a third liquid solvent, thereby forming a silylated amino acid residue of the amino acid H-Apc(X²)—OH, wherein X² is an amino protecting group;
reacting the silylated amino acid residue of the amino acid H-Apc(X²)—OH with an amino acid X³-Phe-Y² in a fourth liquid solvent, thereby producing a peptide fragment of the following formula
X³-Phe-Apc(X²)—OH
or a salt thereof,
wherein X³ is an amino protecting group and Y² is a carboxyl activating group;
reacting the peptide fragment of the following formula
X³-Phe-Apc(X²)—OH,
with an amidating agent in a fifth liquid solvent, thereby producing a peptide fragment of the following formula
X³-Phe-Apc(X²)—NH₂
or a salt thereof;
deprotecting the peptide fragment of the following formula
X³-Phe-Apc(X²)—NH₂,
thereby producing a peptide fragment of the following formula
H-Phe-Apc(X²)—NH₂
or a salt thereof;
deprotecting the peptide fragment of the following formula
X¹-(D)Bal-(D)Trp-OH,
thereby producing the peptide fragment of the following formula
H-(D)Bal-(D)Trp-OH
or a salt thereof;
reacting an amino acid X⁴-Inp-Y³ with the peptide fragment of the following structural formula
H-(D)Bal-(D)Trp-OH,
in a sixth liquid solvent, thereby producing a peptide fragment of the following formula
X⁴-Inp-(D)Bal-(D)Trp-OH
or a salt thereof,
wherein X⁴ is an amino protecting group, and Y³ is a carboxyl activating group;
reacting the peptide fragment of the following formula
X³-Inp-(D)Bal-(D)Trp-OH
with the peptide fragment of the following structural formula
H-Phe-Apc(X²)—NH₂,
in the presence of a nucleophilic additive, in a seventh liquid solvent, thereby producing a peptide fragment of the following formula
X⁴-Inp-(D)Bal-(D)Trp-Phe-Apc(X²)—NH₂
or a salt thereof; and
deprotecting the peptide fragment of the following structural formula
X⁴-Inp-(D)Bal-(D)Trp-Phe-Apc (X²)—NH₂,
thereby producing the peptide fragment of Formula (I)
H-Inp-(D)Bal-(D)Trp-Phe-Apc-NH₂ (I)
or a salt thereof.

17. The process of claim 16, wherein the first through the seventh liquid solvent, each independently, is an organic solvent.

18. The process of claim 2, 4, or 6, wherein the silylating agent is (trimethylsilyl)-N-dimethyl-acetamide.

19. The process of claim 16, wherein the first and the second silylating agents, each, is (trimethylsilyl)-N-dimethyl-acetamide.

20. The process of claim 14 or 16, wherein the nucleophilic additive is selected from the group consisting of 2-hydroxypyridine-N-oxide (HOPO), 1-hydroxy-7-azabenzotriazole (HOAt), 1-hydroxy-benzotriazole (HOBt), 3,4-dihydro-3-hydryoxy-4-oxo-1,2,3-benzotriazine (HODhbt), and ethyl-1-hydroxy-1H-1,2,3-triazole-4-carboxylate (HOCt).

21. The process of claim 20 wherein the nucleophilic additive is 2-hydroxypyridine-N-oxide (HOPO).

22. The process of any one of claim 5, 7, 12, or 16 wherein the carboxyl activating groups Y¹, Y², and Y³, each independently, are selected from the group consisting of N-hydroxysuccinimide (HOSu), N-hydroxyphthalimide, pentafluorophenol (PfpOH), and di-(p-chlorotetrafluorophenyl)carbonate.

23. A peptide fragment of structural formula (II)
Boc-Inp-(D)Bal-(D)Trp-Phe-Apc(Boc)-NH₂, (II)
or a salt thereof.

24. A peptide fragment of structural formula (III)
Boc-Inp-DBal-DTrp-OH, (III)
or a salt thereof.

25. A peptide fragment of structural formula (IV)
H-Phe-Apc(Boc)-NH₂, (IV)
or a salt thereof.

26. A peptide fragment of structural formula (V)
H-DBal-DTrp-OH, (V)
or a salt thereof.

27. A peptide fragment of structural formula (VI)
Z-Phe-Apc(Boc)-NH₂, (VI)
or a salt thereof.
